SEQUENCE LISTING (28 sequences)

SEQ ID NO: 1 - DNA, 2689 bp, synthetic construct
  misc_feature 1..2689: note = Homo sapiens, CCR2 isoform A cDNA
  source 1..2689: mol_type = other DNA
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgaaaaaaaaaaaaaaaaaa

SEQ ID NO: 2 - RNA, 2689 nt, Homo sapiens
  source 1..2689: mol_type = mRNA
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgaaaaaaaaaaaaaaaaaa

SEQ ID NO: 3 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 4 - DNA, 3880 bp, synthetic construct
  misc_feature 1..3880: note = Homo Sapiens, CCR2 isoform B cDNA
  source 1..3880: mol_type = other DNA
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaagatgcattatctatgatatgctaatatatgtatatgcaatatatataggctcttgcttgatctctccaggaggtagtgattatgagaagggggtggagaatgatgagttccttcaccaggagcaaaggacggggatcgtgtggaaccactgcagaactatttccgaaatcaactaagtggagagagccaggaaggctgcatcagaacccagtaaagcttcttgtctggatctgagctggtttgttttgtgcttgcttttccctgccttgccactcccctcactcttctcttttccccacagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttga

SEQ ID NO: 5 - RNA, 3880 nt, Homo sapiens
  source 1..3880: mol_type = mRNA
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaagatgcattatctatgatatgctaatatatgtatatgcaatatatataggctcttgcttgatctctccaggaggtagtgattatgagaagggggtggagaatgatgagttccttcaccaggagcaaaggacggggatcgtgtggaaccactgcagaactatttccgaaatcaactaagtggagagagccaggaaggctgcatcagaacccagtaaagcttcttgtctggatctgagctggtttgttttgtgcttgcttttccctgccttgccactcccctcactcttctcttttccccacagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttga

SEQ ID NO: 6 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 7 - DNA, 21 bp, synthetic construct
  misc_feature 1..21: note = Forward primer; Primer code: C
  source 1..21: mol_type = other DNA
tagggcagtgagagtcatctt

SEQ ID NO: 8 - DNA, 21 bp, synthetic construct
  misc_feature 1..21: note = Forward primer; Primer code: I
  source 1..21: mol_type = other DNA
attcttcggcctgagtaactg

SEQ ID NO: 9 - DNA, 17 bp, synthetic construct
  misc_feature 1..17: note = Forward primer; Primer code: K
  source 1..17: mol_type = other DNA
accctgcttcggtgtcg

SEQ ID NO: 10 - DNA, 20 bp, synthetic construct
  misc_feature 1..20: note = Forward primer; Primer code: P
  source 1..20: mol_type = other DNA
tcagaaataccaacgagagc

SEQ ID NO: 11 - DNA, 20 bp, synthetic construct
  misc_feature 1..20: note = Forward primer; Primer code: R
  source 1..20: mol_type = other DNA
acatctcgttctcggtttat

SEQ ID NO: 12 - DNA, 21 bp, synthetic construct
  misc_feature 1..21: note = Forward primer; Primer code: T
  source 1..21: mol_type = other DNA
attatcactagtaggagagca

SEQ ID NO: 13 - DNA, 20 bp, synthetic construct
  misc_feature 1..20: note = Forward primer; Primer code: W
  source 1..20: mol_type = other DNA
tccttcaccaggagcaaagg

SEQ ID NO: 14 - DNA, 20 bp, synthetic construct
  misc_feature 1..20: note = Forward primer; Primer code: Y
  source 1..20: mol_type = other DNA
aggattgccccactccaaaa

SEQ ID NO: 15 - DNA, 21 bp, synthetic construct
  misc_feature 1..21: note = Forward primer; Primer code: APF
  source 1..21: mol_type = other DNA
gaatcacagtatacgctccat

SEQ ID NO: 16 - DNA, 19 bp, synthetic construct
  misc_feature 1..19: note = Reverse primer: Primer code: D
  source 1..19: mol_type = other DNA
gccagacgtgtgatttcca

SEQ ID NO: 17 - DNA, 21 bp, synthetic construct
  misc_feature 1..21: note = Reverse primer: Primer code: H
  source 1..21: mol_type = other DNA
gtcactccatccactgtctcc

SEQ ID NO: 18 - DNA, 22 bp, synthetic construct
  misc_feature 1..22: note = Reverse primer: Primer code: J
  source 1..22: mol_type = other DNA
gtcatttctgtctctaggctcc

SEQ ID NO: 19 - DNA, 18 bp, synthetic construct
  misc_feature 1..18: note = Reverse primer: Primer code: L
  source 1..18: mol_type = other DNA
agccttcctgcctggtaa

SEQ ID NO: 20 - DNA, 22 bp, synthetic construct
  misc_feature 1..22: note = Reverse primer: Primer code: M
  source 1..22: mol_type = other DNA
cattctttcctggtctcactcc

SEQ ID NO: 21 - DNA, 23 bp, synthetic construct
  misc_feature 1..23: note = Reverse primer: Primer code: N
  source 1..23: mol_type = other DNA
gactttccttttccacgaccatc

SEQ ID NO: 22 - DNA, 20 bp, synthetic construct
  misc_feature 1..20: note = Reverse primer: Primer code: O
  source 1..20: mol_type = other DNA
gctctgccaattgactttcc

SEQ ID NO: 23 - DNA, 20 bp, synthetic construct
  misc_feature 1..20: note = Reverse primer: Primer code: Q
  source 1..20: mol_type = other DNA
gagcaggtaaatgtcagtca

SEQ ID NO: 24 - DNA, 21 bp, synthetic construct
  misc_feature 1..21: note = Reverse primer: Primer code: U
  source 1..21: mol_type = other DNA
tatttctgataaaccgagaac

SEQ ID NO: 25 - DNA, 20 bp, synthetic construct
  misc_feature 1..20: note = Reverse primer: Primer code: V
  source 1..20: mol_type = other DNA
ggtaaatgtcagtcaagcac

SEQ ID NO: 26 - DNA, 20 bp, synthetic construct
  misc_feature 1..20: note = Reverse primer: Primer code: X
  source 1..20: mol_type = other DNA
tgcctggtaacgtagacacc

SEQ ID NO: 27 - DNA, 20 bp, synthetic construct
  misc_feature 1..20: note = Reverse primer: Primer code: Z
  source 1..20: mol_type = other DNA
cctgcctggtaacgtagaca

SEQ ID NO: 28 - DNA, 20 bp, synthetic construct
  misc_feature 1..20: note = Reverse primer: Primer code: APR
  source 1..20: mol_type = other DNA
cattcttaacttggctcgac

(19) United States
(12) Patent Application Publication
(10) Pub. No.: US 2025/0290127 A1
(43) Pub. Date: Sep. 18, 2025

(54) Method And Kit For CCR2 Expression Profiling And Disease Stratification

(71) Applicants: Rutgers, The State University of New Jersey, New Brunswick, NJ (US); Plumeria Therapeutics, Inc., Plainsboro, NJ (US)

(72) Inventors: Lei Yu, Belle Mead, NJ (US); Thomas P. Richardson, West Windsor, NJ (US)

(21) Appl. No.: 19/224,015

(22) Filed: May 30, 2025

Related U.S. Application Data
(63) Continuation of application No. 17/382,240, filed on Jul. 21, 2021, now Pat. No. 12,319,958.
(60) Provisional application No. 63/054,407, filed on Jul. 21, 2020.

(51) Int. Cl. C12Q 1/686; C12Q 1/6851

(52) U.S. Cl. CPC C12Q 1/686; C12Q 1/6851

(57) ABSTRACT

Methods of treating disease include measuring a CCR2 profile of a patent, selecting a CCR2 antagonist based on the CCR2 profile of the patient, and administering the CCR2 antagonist to the patient. Methods for stratifying one or more patients for pain medication based on a CCR2 profile include measuring a CCR2 profile of one or more patients and partitioning the one or more patients into predetermined groups or subgroups based on the CCR2 profile of the one or more patients. Kits for performing the methods are also described.

### RELATED APPLICATION

This application is a continuation of U.S. application Ser. No. 17/382,240, filed on Jul. 21, 2021, which claims the benefit of U.S. Provisional Application No. 63/054,407, filed on Jul. 21, 2020. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 54311005005_2018008_Sequence_Listing.xml; created May 30, 2025, 48,983 Bytes in size.

### BACKGROUND

In assessing pharmacological intervention, a patient population can have a diverse biological makeup, which can impact the pharmacokinetic and pharmacodynamics response to the pharmacological intervention. For example, patients have unique genomes, gene expression profiles, and proteomic profiles, which can influence patient responses to pharmacological treatment.

### SUMMARY

Methods and kits described herein are useful in assessing and stratifying a patient population. Properly stratified using relevant biomarkers, patient populations or subpopulations can be identified that are more responsive to a pharmacological agent. In particular, methods and kits described herein are useful in stratifying a patient population with respect to responsiveness to C-C chemokine receptor type 2 (CCR2) antagonists.

Described herein is a method for treating a disease in a patient in need thereof. The method can include measuring a CCR2 profile of the patient, selecting a compound based on the CCR2 profile of the patient, and administering the CCR2 antagonist to the patient. The compound can be a CCR2 antagonist, agonist, or mixed agonist. The CCR2 profile can include a profile of CCR2 isoform A, CCR2 isoform B, or both. Each CCR2 isoform can include one or more respective polymorphisms. The compound can be an antagonist or agonist of at least one polymorphism identified from the measured CCR2 profile of the patient.

Measuring the CCR2 profile can include sequencing CCR2 proteins or nucleic acids within the patient. Measuring the CCR2 profile can include sequencing nucleic acids within the patient. The nucleic acids can be DNA. The nucleic acids can be mRNA, such as mRNA within a bodily fluid (e.g., blood) of the patient. The method can further include separating a biological material based on the sequenced proteins or nucleic acids. The biological material can include cells, tissue, blood, other bodily fluids, or a combination thereof.

Measuring the CCR2 profile of the patient can include measuring a CCR2 profile of one or any combination of the patient's cells, the patient's tissues, the patient's blood, other bodily fluids of the patient. Measuring the CCR2 profile of the patient can include measuring a CCR2 profile of the patient's cells, optionally wherein the cells are a plurality of cell types. Measuring the CCR2 profile of the patient of the patient can include measuring a CCR2 profile of the patient's tissues, optionally wherein the tissues are a plurality of tissue types. Measuring the CCR2 profile of the patient can include measuring a CCR2 profile of the patient's other bodily fluids, optionally wherein the other bodily fluids are a plurality of types of bodily fluids. Measuring the CCR2 profile of the patient can include measuring a protein expression level for CCR2 isoform A, CCR2 isoform B, or both. The method can further include separating a biological material based on the measured protein expression level for CCR2 isoform A, CCR2 isoform B, or both. The biological material can include cells, tissue, blood, other bodily fluids, or a combination thereof.

Measuring a CCR2 profile can include subjecting a biological material to one or more compounds. The biological material can include cells, tissue, blood, other bodily fluids, or a combination thereof. The one or more compounds can be antagonists or agonists of at least one polymorphism identified from the measured CCR2 profile of the patient. The method can further include measuring one or more of biochemical, molecular biological, and cellular biological changes in the biological material prior to and after subjecting the biological material to the one or more compounds. Measuring a CCR2 profile can include obtaining a biological sample from the patient.

The compound can be one or more of INCB-8761/PF-4136309, MK-0812, BMS-813160, INCB-003284, PF-04634817, BMS-741672, Cenicriviroc (CCR2+CCR5), and CCX-140. Also described is a kit for performing any of such methods, where the kit can include one or more reagents for measuring a CCR2 profile of the patient.

The selected compound can be an antagonist of at least one polymorphism of CCR2 isoform A identified from the measured CCR2 profile of the patient. The selected compound can be an agonist of at least one polymorphism of CCR2 isoform A identified from the measured CCR2 profile of the patient. The selected compound can be an antagonist of at least one polymorphism of CCR2 isoform B identified from the measured CCR2 profile of the patient. The selected compound can be an agonist of at least one polymorphism of CCR2 isoform B identified from the measured CCR2 profile of the patient. The selected compound can be one of either an antagonist or an agonist for each of a plurality of polymorphisms of CCR2 isoform A identified from the measured CCR2 profile of the patient. The selected compound can be an antagonist for each of a plurality of polymorphisms of CCR2 isoform A identified from the measured CCR2 profile of the patient. The selected compound can be an agonist for each of a plurality of polymorphisms of CCR2 isoform A identified from the measured CCR2 profile of the patient. The selected compound can be one of either an antagonist or agonist for each of a plurality of polymorphisms of CCR2 isoform B identified from the measured CCR2 profile of the patient. The selected compound can be an antagonist for each of a plurality of polymorphisms of CCR2 isoform B identified from the measured CCR2 profile of the patient. The selected compound can be an agonist for each of a plurality of polymorphisms of CCR2 isoform B identified from the measured CCR2 profile of the patient. The selected compound can be an agonist of at least one polymorphism of CCR2 isoform A identified from the measured CCR2 profile of the patient and an antagonist of at least one polymorphism of CCR2 isoform B identified from the measured CCR2 profile of the patient. The selected compound can be an antagonist of at least one polymorphism of CCR2 isoform A identified from the measured CCR2 profile of the patient and an agonist of at least one polymorphism of CCR2 isoform B identified from the measured CCR2 profile of the patient.

Described herein is a method for stratifying one or more patients for pain medication based on a CCR2 profile. The method can include measuring a CCR2 profile of one or more patients and partitioning the one or more patients into predetermined groups or subgroups based on the CCR2 profile of the one or more patients. The CCR2 profile can include a profile of CCR2 isoform A, CCR2 isoform B, or both. Each CCR2 isoform can include one or more respective polymorphism.

Measuring the CCR2 profile of the one or more patients can include sequencing CCR2 proteins or nucleic acids within the patient. The nucleic acids can be DNA. The nucleic acids can be mRNA, such as mRNA within a bodily fluid (e.g., blood) of the patient. The method can further include separating a biological material based on the sequenced proteins or nucleic acids. The biological material can include cells, tissue, blood, other bodily fluids, or a combination thereof.

Measuring the CCR2 profile of the one or more patients can include measuring a CCR2 profile of one or any combination of the patient's cells, the patient's tissues, the patient's blood, and other bodily fluids of the patient. Measuring the CCR2 profile of the one or more patients can include measuring a CCR2 profile of the patient's cells, optionally wherein the cells are a plurality of cell types. Measuring the CCR2 profile of the one or more patients can include measuring a CCR2 profile of the patient's tissues, optionally wherein the tissues are a plurality of tissue types. Measuring the CCR2 profile of the one or more patients can include measuring a CCR2 profile of the patient's other bodily fluids, optionally wherein the other bodily fluids are a plurality of types of bodily fluids. Measuring the CCR2 profile of the one or more patients can include measuring protein expression level for CCR2 isoform A, CCR2 isoform B, or both. The method can further include separating a biological material based on the measured protein expression level for CCR2 isoform A, CCR2 isoform B, or both.

Measuring a CCR2 profile can include subjecting a biological material to one or more compounds. The biological material can include cells, tissue, blood, other bodily fluids, or a combination thereof. The one or more compounds can be antagonists or agonists of at least one polymorphism of identified from the measured CCR2 profile of the patient. The method can further include measuring one or more of biochemical, molecular biological, and cellular biological changes in the biological material prior to and after subjecting the cells to the one or more compounds. Measuring a CCR2 profile can include obtaining a biological sample from the patient.

The CCR2 one or more compounds can be one or more of INCB-8761/PF-4136309, MK-0812, BMS-813160, INCB-003284, PF-04634817, BMS-741672, Cenicriviroc (CCR2+CCR5), and CCX-140.

The method can further include identifying statistically significant differences among patient groups or subgroups. Also described is a kit for performing any of such methods, where the kit can include one or more reagents for measuring a CCR2 profile of the patient. The CCR2 profile can include a profile of CCR2 isoform A, CCR2 isoform B, or both. Each CCR2 isoform can include one or more respective polymorphisms. The one or more reagents comprises a forward primer comprising a sequence selected from SEQ ID NOs: 7 through 15 and a reverse primer comprising a sequence selected from SEQ ID NOs: 16 through 28.

Described herein is a method for generating a profile of CCR2A and CCR2B isoform expression levels for a human subject. The method includes: a) obtaining a sample from the human subject; b) extracting RNA from the sample; c) performing reverse transcription on the extracted RNA to generate cDNA; d) amplifying the cDNA by performing polymerase chain reaction (PCR) by using a forward primer comprising a sequence selected from SEQ ID NOs: 7 through 15 and a reverse primer comprising a sequence selected from SEQ ID NOs: 16 through 28; and e) quantifying CCR2A and CCR2B isoform expression levels from the amplified cDNA.

Quantifying CCR2A and CCR2B isoform expression levels can be performed in a variety of ways, including by gel densitometry, blotting, nucleic acid hybridization, fluorescent in situ hybridization, reverse transcription-polymerase chain reaction, real-time polymerase chain reaction, microarray, nucleic acid sequencing, enzyme-linked immunosorbent assay (ELISA), colorimetric quantification, or other optical methods.

The sample can be blood. The sample can be a bodily secretion, such as saliva, urine, nasal mucosa, or vaginal mucosa.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a human CCR2 isoform A (SEQ ID NO: 3) vs. B (SEQ ID NO: 6) protein sequence comparison.

FIG. 2 is a human CCR2 isoform A (SEQ ID NO: 1) vs. B (SEQ ID NO: 4) cDNA sequence comparison.

FIG. 3 is a graph of human CCR2 isoform A transmembrane helices.

FIG. 4 is a graph of human CCR2 isoform B transmembrane helices.

FIG. 5 is a chart of CCR2 polymorphisms identified by rs number.

FIG. 6 is an illustration of a kit for performing methods described herein.

FIGS. 7A-C are graphs of ratios of human CCR2 isoforms in human subjects. FIG. 7A is the CCR2A/2B ratio of three human subjects with diabetes. FIG. 7B is the CCR2A/2B ratio of eight human subjects with diabetic neuropathy. FIG. 7C is the CCR2A/2B ratio of six normal human subjects without diabetes-related disease.

### DETAILED DESCRIPTION

A description of example embodiments follows.

As used herein, an “agonist” is an agent (e.g., compound) that binds to a receptor and activates the receptor to produce a biological response, such as an increase or decrease in expression or activity of a target gene, protein, or a pathway. An agonist can also increase the activity of a pathway through modulating the activity of pathway components, for example, through inhibiting the activity of negative regulators of a pathway. For example, an agonist of CCR2 isoform A is an agent that binds to the C-C chemokine receptor type 2 isoform A. An agonist of CCR5 is an agent that binds to the C-C chemokine receptor type 5. As used herein, an “agonist” encompasses an “inverse agonist,” which is an agent (e.g., a compound) that binds to the same receptor as a reference agonist, but induces an opposite biological response relative to the reference agonist. As used herein, an “agonist” encompasses a “partial agonist,” which is an agent (e.g., a compound) that binds to the same receptor as a reference agonist, but produces a biological response having only partial efficacy relative to the reference agonist. As used herein, an “agonist” encompasses a “partial inverse agonist,” which is an agent (e.g., a compound) that binds to the same receptor as a reference agonist, but induces an opposite biological response relative to the reference agonist and having only partial efficacy relative to a reference inverse agonist. As used herein, an “antagonist” is an agent (e.g., compound) that binds to a receptor and dampens a biological response by binding to and blocking a receptor rather than activating it like an agonist. As used herein, an “antagonist” encompasses a “partial antagonist,” which is an agent (e.g., a compound) that binds to a receptor and partially dampens a biological response. Whether a compound is an agonist, an inverse agonist, a partial agonist, a partial inverse agonist, or an antagonist can be measured by methodologies to detect molecular interactions such as proximity determination analysis. In another example of embodiment, cellular activities at the biochemical and/or physiological levels are determined as a measure of an agent's agonist activity, either in single cells, or in cell-cell interactions, or both. In yet another example of embodiment, a chemical indicator or indicators, such as fluorescent indicator compounds and/or luciferase markers, are introduced into an assay, and an agent's agonist activity is determined by measuring the said chemical indicators' properties. In yet another example of embodiment, one or more chemical indicators, one or more cellular types, or a mixture of chemical indicators and cellular types, are placed on a containment, said containment consisting of paper, cloth, plastic or other forms of polymers, metal, or a combination thereof; then an agent and said containment are enabled to be in contact, and an agent's agonist activity is determined by measuring the said chemical indicators' properties and/or cellular activities. In yet another example of embodiment, an agonist to be measured is in a purified form, or in a mixture of liquid, solid, and/or gas state, and is enabled to be in contact with a containment described above, for the purpose of measuring an agonist's activity.

Whether a compound is an agonist or antagonist, and the degree of agonism or antagonism, can be determined and measured by methodologies that detect molecular interactions such as proximity determination analysis. In another example embodiment, cellular activities at the biochemical and/or physiological levels are determined as a measure of an agent's agonist activity, either in single cells, or in cell-cell interactions, or both. In yet another example embodiment, a chemical indicator or indicators, such as fluorescent indicator compounds and/or luciferase markers, are introduced into an assay, and an agent's agonist activity is determined by measuring the said chemical indicators' properties. In yet another example embodiment, one or more chemical indicators, one or more cellular types, or a mixture of chemical indicators and cellular types, are placed on a containment, said containment consisting of paper, cloth, plastic or other forms of polymers, metal, or a combination thereof; then an agent and said containment are enabled to be in contact, and an agent's agonist activity is determined by measuring the said chemical indicators' properties and/or cellular activities. In yet another example embodiment, an agonist to be measured is in a purified form, or in a mixture of liquid, solid, and/or gas state, and is enabled to be in contact with a containment described above, for the purpose of measuring an agonist's activity.

In some instances, a compound can be an agonist of a CCR2 isoform and also an antagonist of another CCR2 isoform, and vice-versa. In some instances, a compound can be an agonist of a CCR2 isoform and also an antagonist of another CCR type, such as CCR5. Compounds exhibiting any of these characteristics can be referred to as a “mixed agonist-antagonist” or a “mixed antagonist-agonist.”

Genetic variations exist in human beings, making each human being unique at the genetic level. Even for identical twins, the exact gene activity levels are different. Pharmaceutical compounds, when utilized to treat certain pathological conditions, produce varied treatment results in different individuals. A contributing factor to such inter-individual responses to drug treatment is likely genetic differences among individuals.

Described herein are methods and kits for use in stratifying members of a population (e.g., member of a patient population, preferably a human patient population) based on their specific genetic profiles of a given biological system, particularly the C-C chemokine receptor 2 (CCR2). Stratifying a patient population can be useful in a variety of clinical studies, including, without limitation, in prospective and retrospective cohort studies. For example, it can be desirable to study an association between an outcome variable (e.g., a clinical endpoint, such as degree of pain remission) and an exposure variable (e.g., treatment with a given dose of a given CCR2 receptor antagonist). In such a study, the strength of correlation between the exposure variable and the outcome variable can be masked, changed, altered, or confounded by relevant, though unaccounted-for, CCR system heterogeneities among patients. Stratifying a patient population (e.g., according to relevant CCR system markers, characteristics, genotypes, or phenotypes) prior to statistical analysis could aid in refining these relationships, and therefore in conducting clinical studies. Some CCR2 antagonists are likely to be more or less effective than others for individuals in different CCR2 strata. This could in turn improve drug treatment outcomes, as drugs could be administrated to patients with specific genetic profiles, based on knowledge of the relationship between that genetic type and efficacy of treatment with a given CCR2 antagonist.

The C-C chemokine receptor (CCR) system is highly complex and has been studied in academia and industry in efforts to elucidate the role of individual components in many distinct diseases marked by underlying inflammation. Some of these diseases include multiple sclerosis, rheumatoid arthritis, osteoarthritis, chronic obstructive pulmonary disease, chronic pain, and others. However, the CCR biology is insufficiently understood to enable successful clinical interventions. The methods and kits described herein address a large gap in the scientific understanding of the CCR system based on a detailed understanding of the genetic and expression variability among patients and disease states. As a result, more successful clinical development strategies can be formulated. The chemokine network is known to have at least 20 receptors and 80 ligands and displays promiscuity. CCR2 (aka CD192) may bind CCL2 (MCP1), CCL7 (MCP3), CCL8 (MCP2), CCL13 (MCP4), and CCL16.

The C-C chemokine receptor type 2 (CCR2) protein is encoded by the CCR2 gene. CCR2 is a member of a large family of inflammatory mediators and there is considerable pre-clinical evidence of its role in contributing to pain. Several knock out (KO) mice studies and in vivo models (e.g., chronic constriction injury (CCI) and spinal nerve ligation) demonstrated the role of CCR2 in pain.

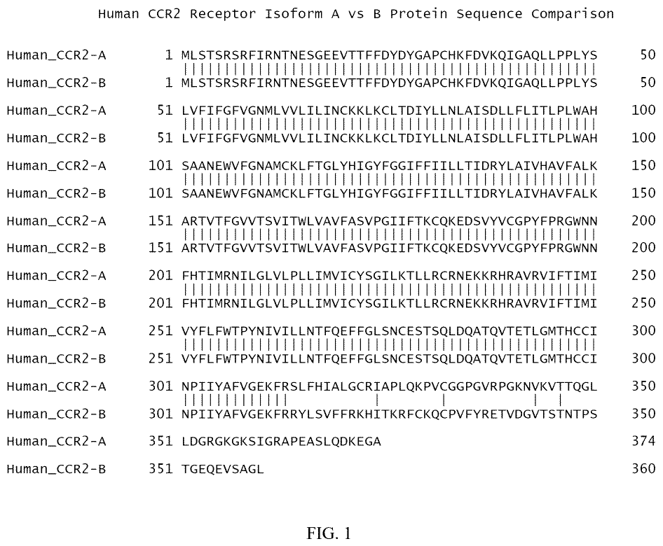

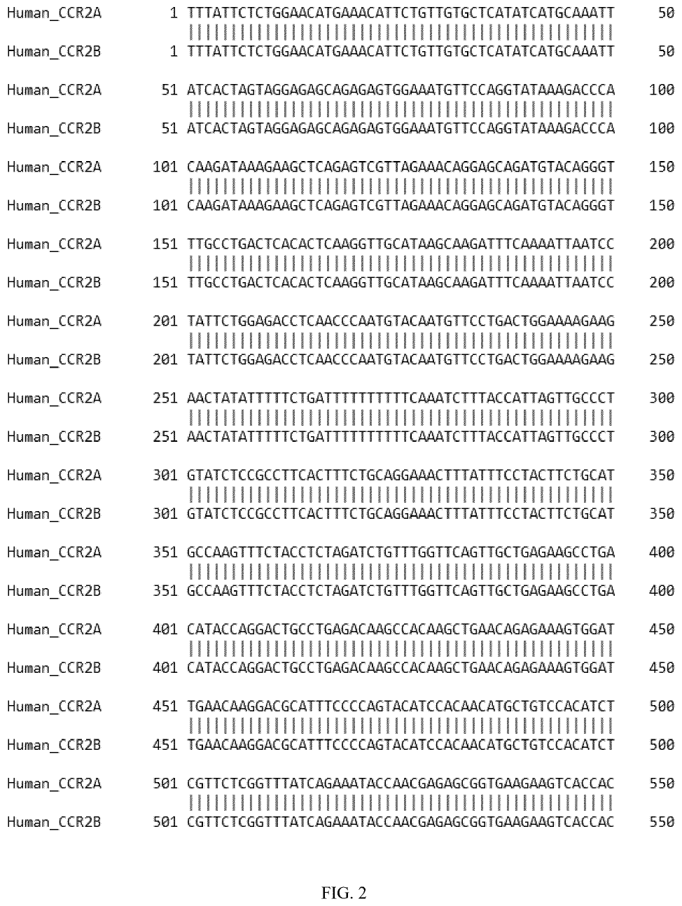

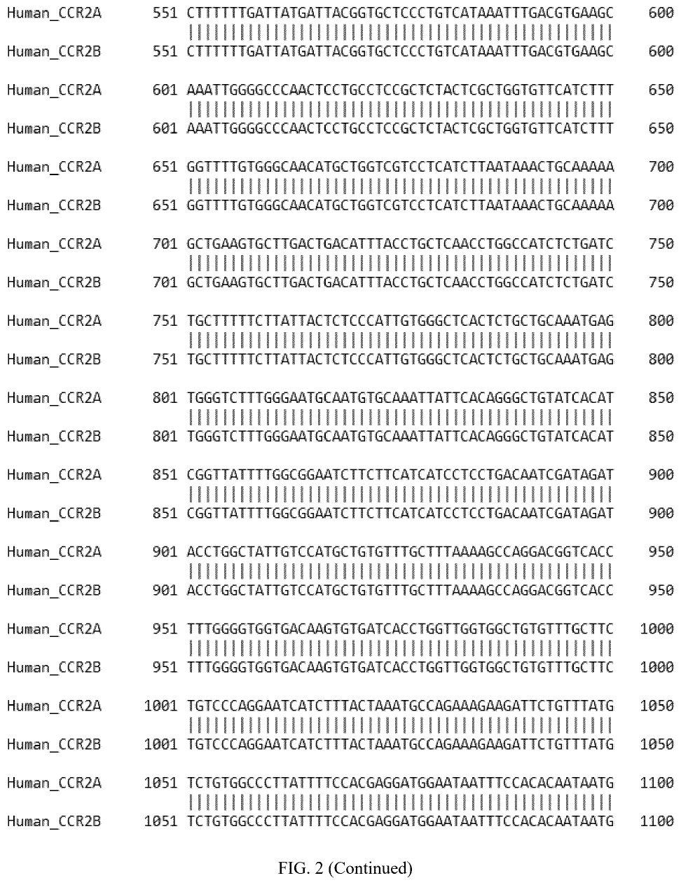

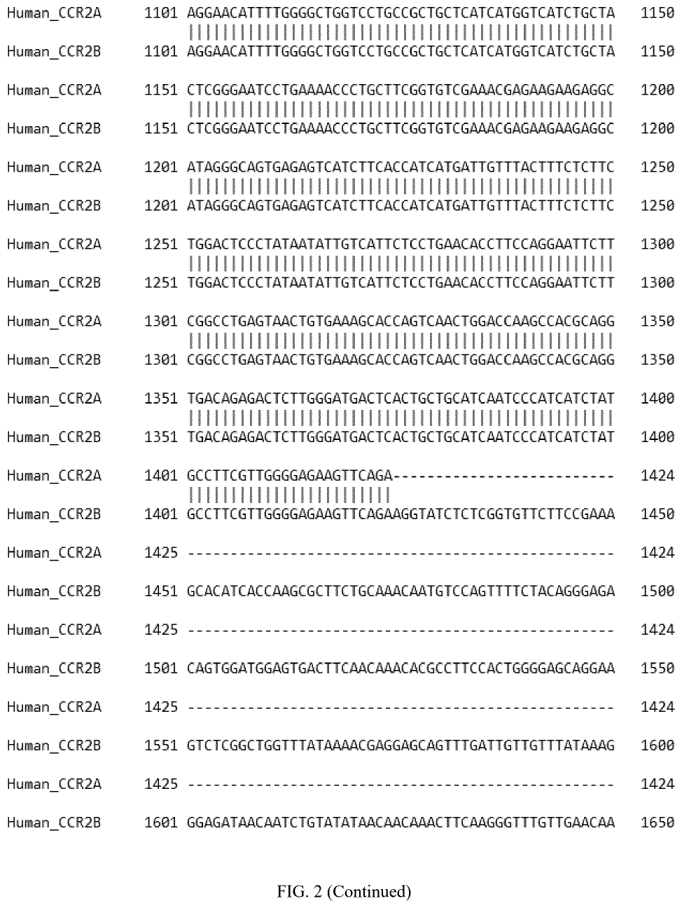

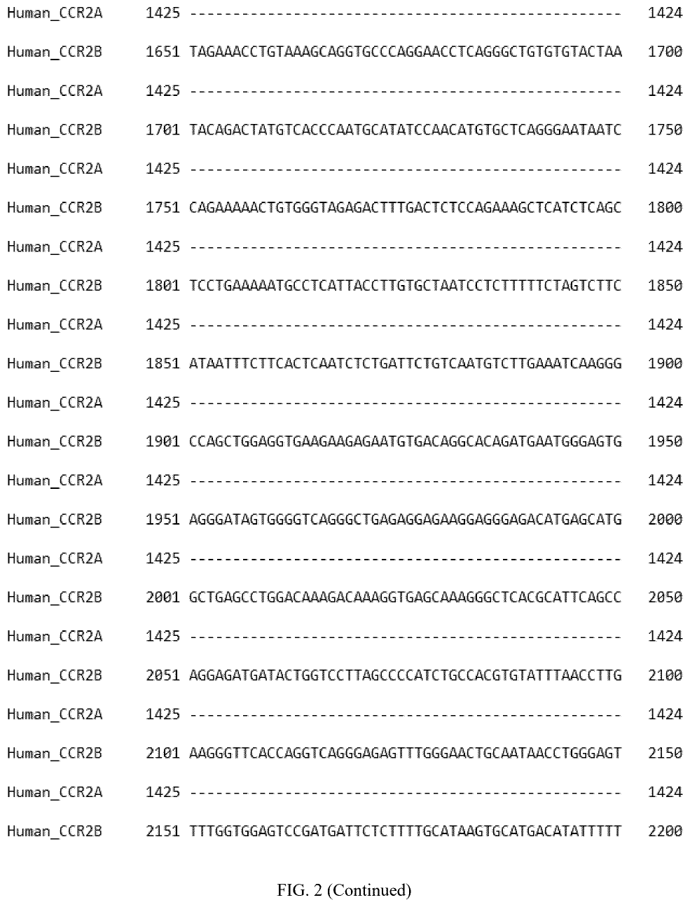

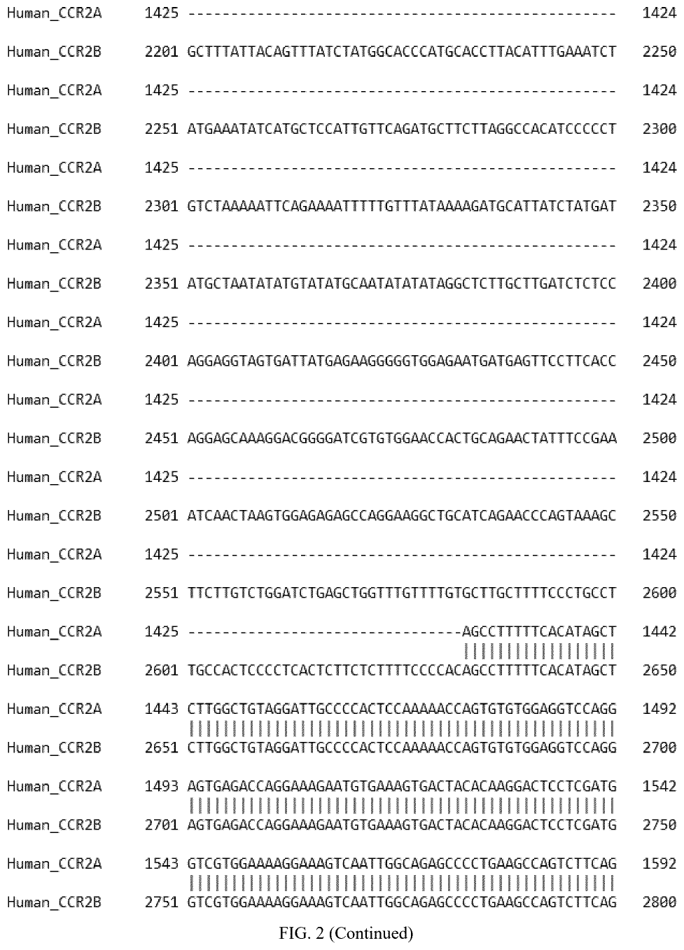

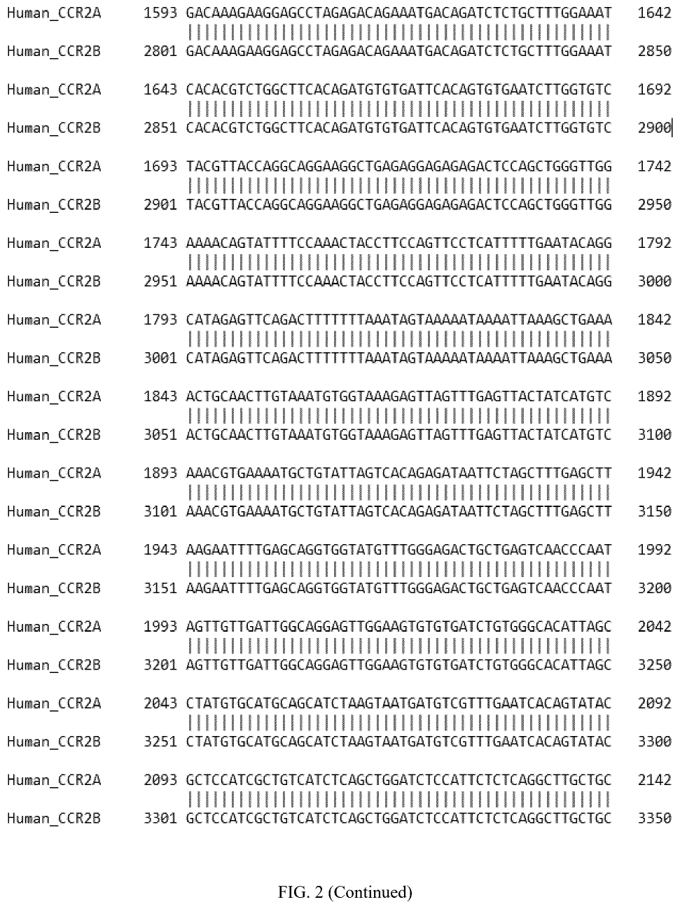

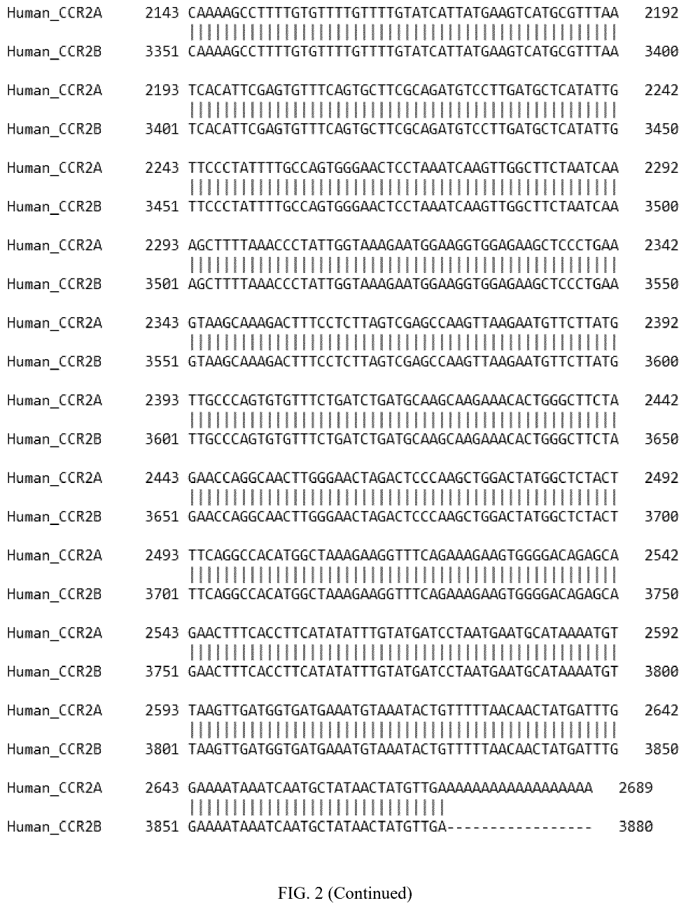

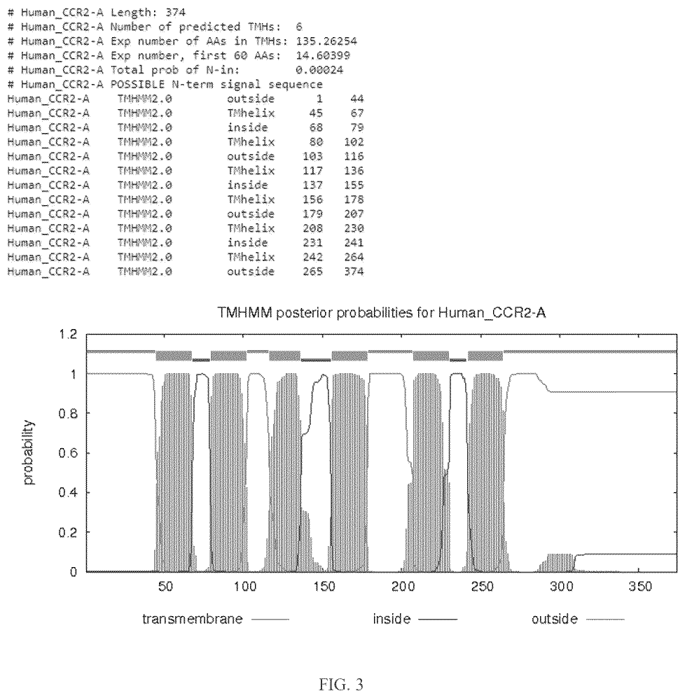

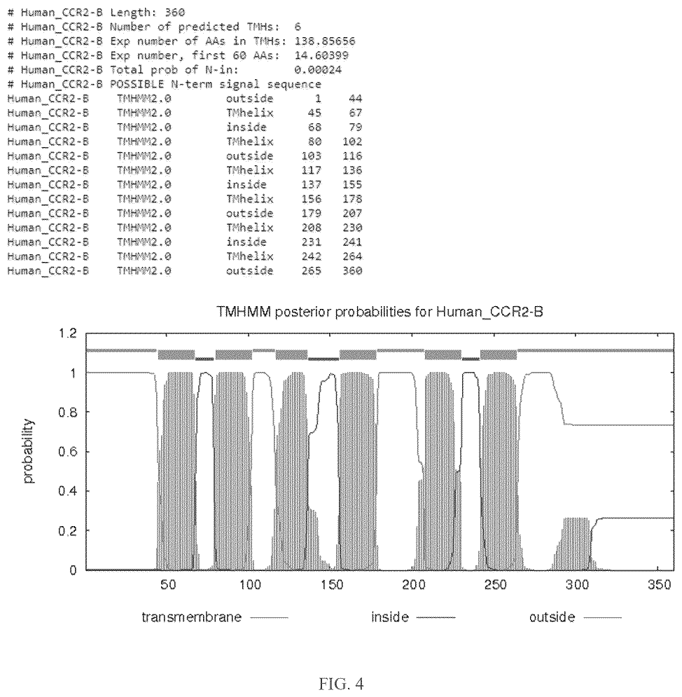

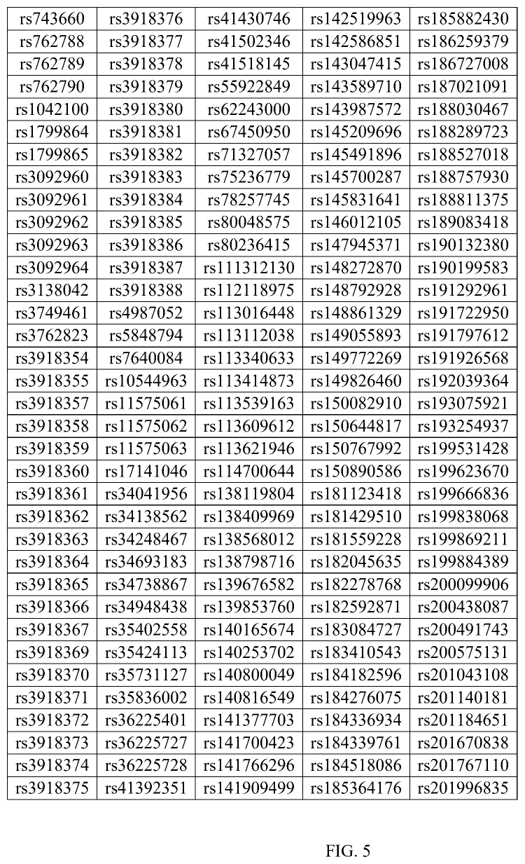

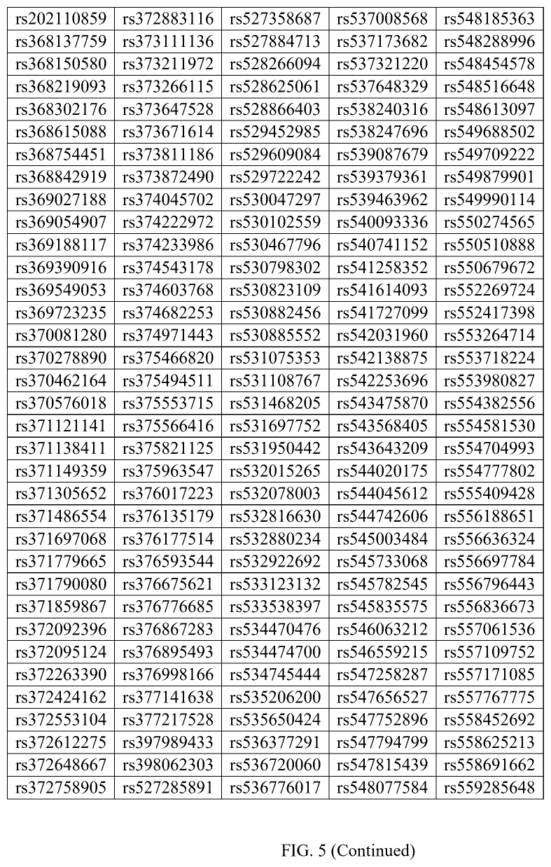

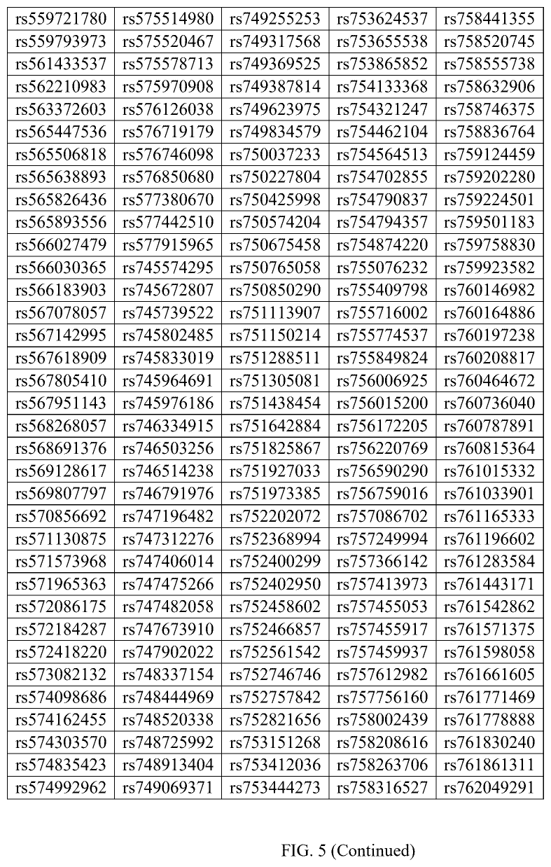

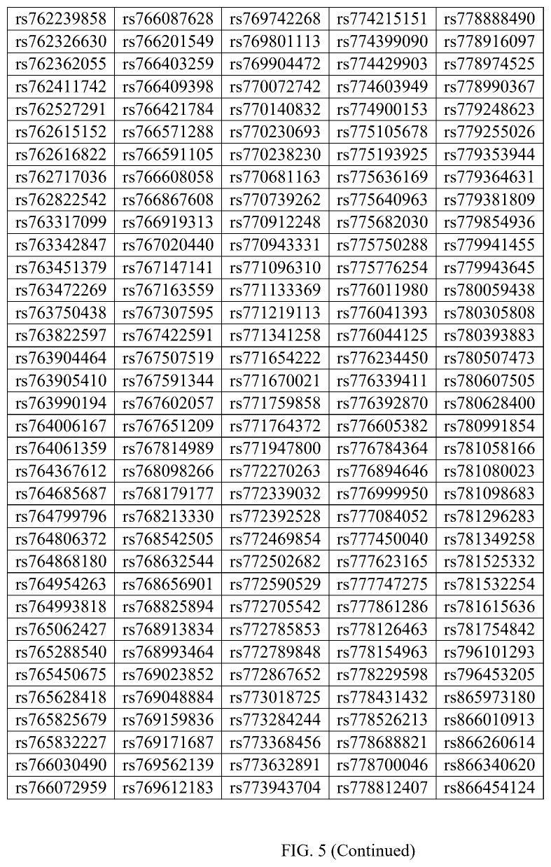

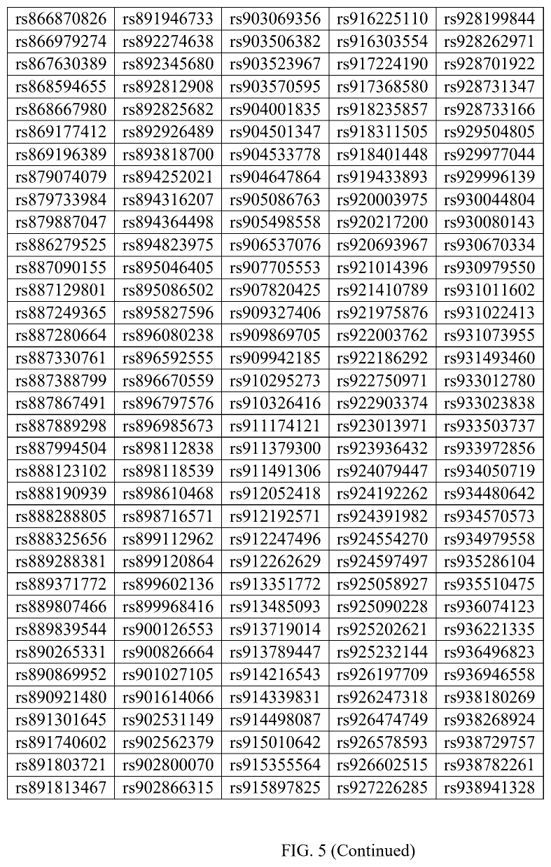

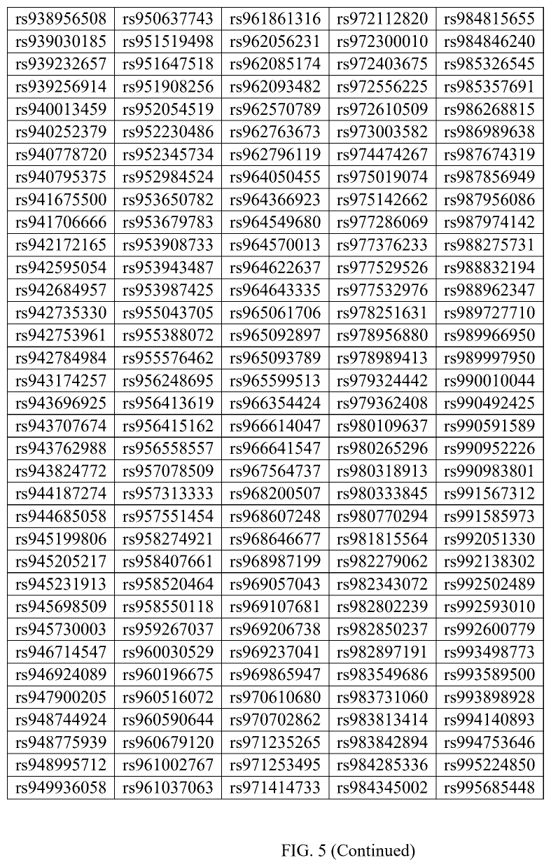

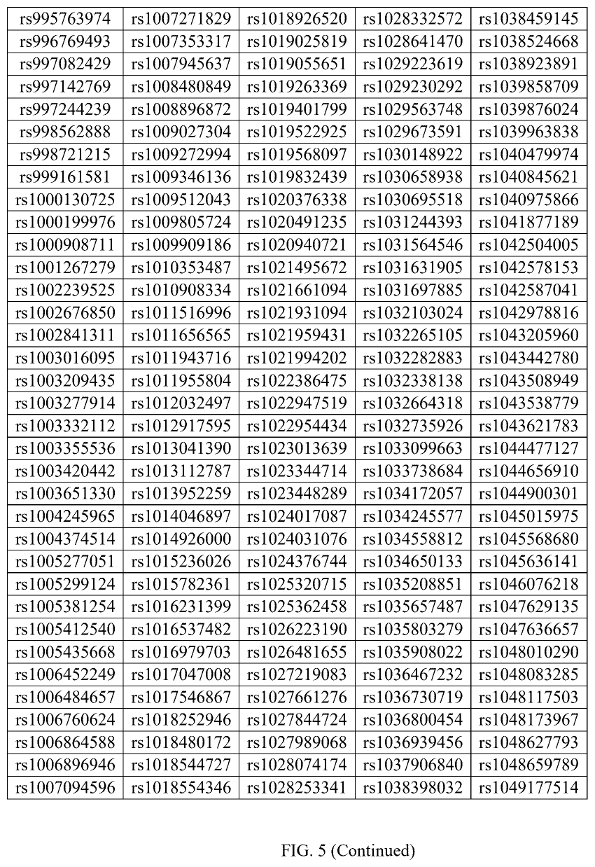

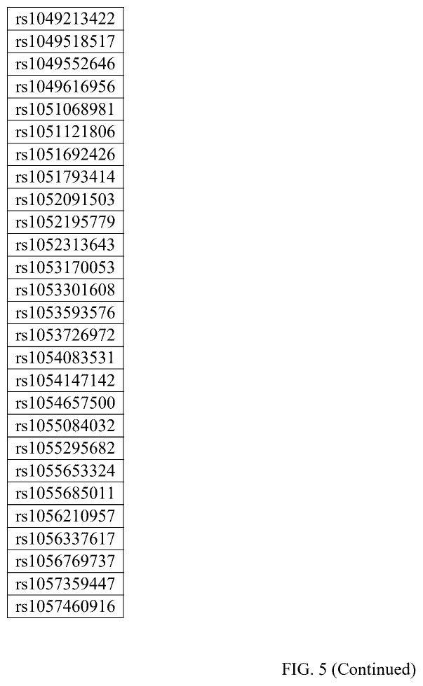

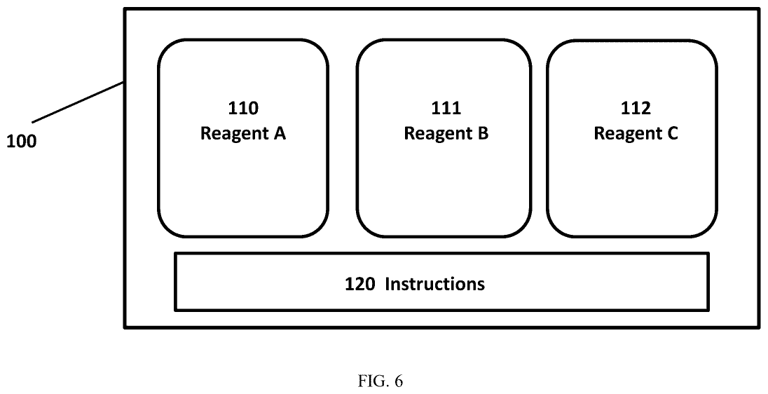

CCR2 exhibits substantial differences at the genetic and protein level. At least two alternatively spliced transcript variants are known to exist. As shown in the CCR2 isoform A vs. B protein (FIG. 1) and mRNA (FIG. 2) sequence comparisons, a major portion of CCR2 isoforms A and B is identical, starting at the 5′ end of the CCR2 mRNA, and at the amino terminus of the CCR2 protein. However, sequence divergence is striking closer to the carboxyl terminus portion of the CCR2 protein (corresponding to the 3′ portion of the CCR2 mRNA), with little sequence homology at either the protein level or the mRNA level. In addition, over 1,200 polymorphisms have been reported for the CCR2 gene region. FIG. 2 is the primary sequence information for the two isoforms, highlighting the specific differences in the receptor A and B isoforms at the genetic level. FIGS. 3 and 4 are hydrophobicity plots for CCR2A and CCR2B, respectively, showing the predicted transmembrane regions of the receptors and illustrating the differences at the terminal ends that may confer some of the sequence-dependent functionalities of the receptors. FIG. 6 is a chart of CCR2 polymorphisms identified by rs number. The rs number refers to the reference single nucleotide polymorphism (SNP) ID number. These sequence diversities form the basis of CCR2 structural and functional variations.

CCR2 is also known to have A and B isoforms, and heterodimerization may occur. CCR2 may also heterodimerize with other C-C chemokine receptors (CCRs), such as CCR5. Additionally, different tissues display different expression profiles, which can also vary depending on levels of stress. A profile of these factors can represent a biochemical signature of an individual patient and provide indicia of anticipated response to CCR2 antagonists.

Patients diagnosed with different diseases may exhibit differences in CCR2 profiles. By evaluating CCR2 profiles, a patient population can be stratified based on genotype and phenotype. In general, stratification refers to partitioning a patient population into groups or subgroups based on particular genotypic or phenotypic markers. Stratification is based on assessing a combination of factors, including: 1) the specific genetic profile of CCR2 isoforms, A and/or B, as determined by the alternative splicing of expressed variants (A and/or B); 2) genomic variations within an individual's polymorphisms; 3) protein expression level (e.g., ng/ml or pg/mL) for each A and B isoform; 4) identity of the specific tissue expressing CCR2 A and/or B isoforms; and 5) changes in CCR2 protein expression and/or CCR2 mRNA expression from baseline under various forms of stress, such as acute and chronic injury and neuroinflammation, and in chronic diseases, such as diabetes, osteoarthritis, multiple sclerosis, rheumatoid arthritis, and diabetic neuropathy. Different population groups are expected to have different responses, some more favorable than others, to pharmacologic intervention with CCR2 antagonists, agonists, or mixed agonist-antagonists. For example, particular antagonists may show greater efficacy when administered to particular patient populations or subpopulations diagnosed with particular clinical indications, thereby providing improved methods of treatment. By stratifying a patient population based on appropriate factors and documenting individual patient profiles, clinical trials can be improved, for example, by providing CCR2 antagonists (or agonists or mixed agonist-antagonists) to particular patient groups or subgroups expected, or anticipated, to have improved response to one or more CCR2 antagonists (or agonists or mixed agonist-antagonists).

Examples of CCR2 antagonists include INCB-8761/PF-4136309, MK-0812, BMS-813160, INCB-003284, PF-04634817, BMS-741672, Cenicriviroc (CCR2+CCR5), and CCX-140.

INCB-8761 and PF-4136309 refer to a compound having the following structure:

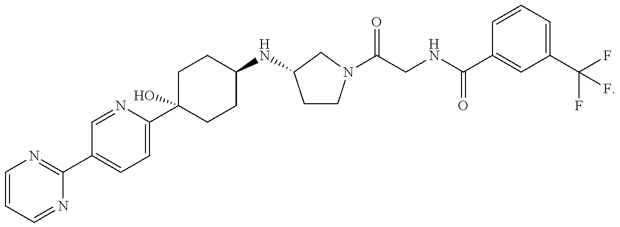 [SMILES: O=C(NCC(=O)N1CC[C@H](N[C@H]2CC[C@@](O)(c3ccc(-c4ncccn4)cn3)CC2)C1)c1cccc(C(F)(F)F)c1]

MK-0812 refers to a compound having the following structure:

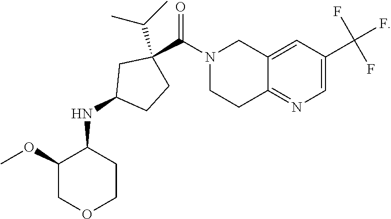 [SMILES: CO[C@@H]1COCC[C@@H]1N[C@@H]1CC[C@@](C(=O)N2CCc3ncc(C(F)(F)F)cc3C2)(C(C)C)C1]

BMS-813160 refers to a compound having the following structure:

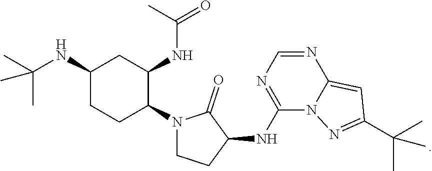 [SMILES: CC(=O)N[C@@H]1C[C@H](NC(C)(C)C)CC[C@@H]1N1CC[C@H](Nc2ncnc3cc(C(C)(C)C)nn23)C1=O]

INCB-003284 refers to a compound having the following structure:

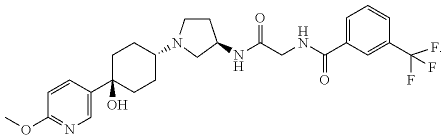 [SMILES: COc1ccc([C@]2(O)CC[C@H](N3CC[C@@H](NC(=O)CNC(=O)c4cccc(C(F)(F)F)c4)C3)CC2)cn1]

PF-04634817 refers to a compound having the following structure:

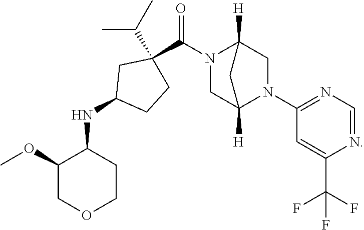 [SMILES: CO[C@@H]1COCC[C@@H]1N[C@@H]1CC[C@@](C(=O)N2C[C@@H]3C[C@H]2CN3c2cc(C(F)(F)F)ncn2)(C(C)C)C1]

BMS-741672 refers to a compound having the following structure:

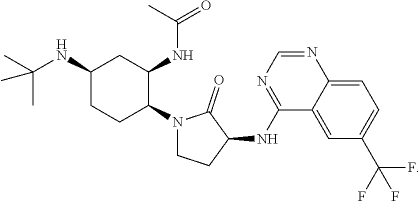 [SMILES: CC(=O)N[C@@H]1C[C@H](NC(C)(C)C)CC[C@@H]1N1CC[C@H](Nc2ncnc3ccc(C(F)(F)F)cc23)C1=O]

Cenicriviroc (CCR2+CCR5) refers to a compound having the following structure:

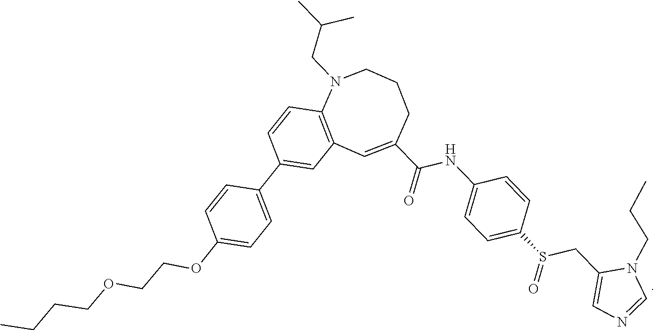 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1]

CCX-140 refers to a compound having the following structure:

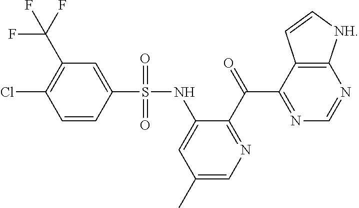 [SMILES: Cc1cnc(C(=O)c2ncnc3[nH]ccc23)c(NS(=O)(=O)c2ccc(Cl)c(C(F)(F)F)c2)c1]

CCR2 antagonists, agonists, and mixed agonist-antagonists can be administered to patients suffering from pain associated with, or resulting from, a variety of diseases and disorders. Examples of particular diseases and disorders are described in WO 2010/071567 at pages 9-13, numbered paragraphs 1-15. The entire contents of pages 9-13, numbered paragraphs 1-15 of WO 2010/071567 is incorporated by reference herein in its entirety. Some examples of diseases and disorders for which pain can be treated by administration of a CCR2 antagonist include neuroinflammation and pain linked to CCR2. More particularly, pain can be associated with complex regional pain syndrome, neurofibromatosis, sarcoidosis, ankylosing spondylitis, postherpetic neuralgia, and painful diabetic neuropathy. In addition, pain can be associated with multiple sclerosis, rheumatoid arthritis, osteoarthritis, human immunodeficiency virus (HIV), and acquired immunodeficiency syndrome (AIDS).

The methods described herein utilize a genotyping approach to understand the genetic polymorphisms and gene expression profiles within a patient population to enable continued, and targeted, clinical development. Specifically, aspects of the invention relate to elucidating the molecular profile of CCR2 genotypes and isoforms, for determining the key pharmacological, biochemical, and stoichiometric parameters of the receptors, for assessing the acute and chronic desensitization of the receptors, and for measuring dosing and competition with ligand and test compound. The results will provide important clues to enable the design and implementation of clinical development strategies for targeting this important receptor.

Briefly, scientific validation involves the following steps, which are described in more detail in subsequent paragraphs: 1) Obtaining biological samples from a human patient population. Preferably, the biological samples have cells that express the CCR2 chemokine receptor. 2) Measuring CCR2 genotype variations (polymorphisms) and expressed receptor mRNA diversity (receptor isoforms). 3) Separating cells based on said genotype polymorphisms and/or mRNA diversity; cells in this step may be either cells in said biological samples, or cells containing said polymorphisms/mRNA diversity that are constructed using molecular genetics techniques. 4) Subjecting said cells to one or more chemical compounds that display CCR2 affinity, such as CCR2 antagonists, agonists, or mixed agonist-antagonists. 5) Measuring biochemical, molecular biological, and/or cellular biological changes in said cells both without and with said chemical compound exposure. 6) Performing analysis to determine if said changes represent statistically significant differences; statistically significant differences from such analysis validate the method for patient stratification, at a pre-clinical therapeutic intervention level.

Measuring CCR2 genotype variations (polymorphisms) can include sequencing nucleic acids (e.g., DNA and/or RNA) from individual's biological material (e.g., cells, tissue, blood, other bodily fluids, or a combination thereof), subjecting the nucleic acids to pre-formed templates such as microarray chips to quantify the extent and the nature of genotype variations, and deducing genotype variations by a variety of molecular detection methods such as mass spectrometry/spectroscopy, electrophoresis, and marker-mediated sorting.

Measuring expressed receptor mRNA diversity can include sequencing mRNA from an individual's biological material (e.g., cells, tissue, blood, other bodily fluids, or a combination thereof), either directly or by first converting said mRNA into cDNA form, subjecting mRNA to pre-formed templates such as microarray chips to quantify the extent and the nature of mRNA diversity, and deducing mRNA diversity by a variety of molecular detection methods such as mass spectrometry/spectroscopy, electrophoresis, and marker-mediated sorting. In some instances, mRNA is obtained from a blood sample of a patient.

Separating cells based on said genotype polymorphisms and/or mRNA diversity can include cell staining, marker-mediated sorting, density differentiation, morphology differentiation, biochemical differentiation, and functional differentiation.

Subjecting said biological material (e.g., cells, tissue, blood, other bodily fluids, or a combination thereof) to one or more chemical compounds that display CCR2 affinity can include direct exposure of cells to said chemical compounds in a variety of milieu, or indirect exposure of cells to said chemical compounds by placing cells in conditions where said chemical compounds have been a component thereof.

Measuring biochemical, molecular biological, and/or cellular biological changes in said biological material (e.g., cells, tissue, blood, other bodily fluids, or a combination thereof) both without and with said chemical compound exposure can include exposing said biological material to a variety of milieu containing said chemical compound or another compound serving as a comparison reference, then measuring desirable biochemical, molecular biological, and/or cellular biological changes in said cells either directly by physical, chemical, or other means, or indirectly by first obtaining certain cellular components from said biological material, then measuring biochemical, molecular biological, and/or cellular biological changes in said cellular components.

Other bodily fluids can include, for example, saliva, nasal mucus, sweat, cerebrospinal fluid, lymphoid fluid, vaginal secretion, rectal secretion, and urine.

Commercial validation can involve the following steps: A) Separating human patients into groups based on the above-described pre-clinical level stratification. B) Treating said human subject groups with CCR2 compounds (e.g., CCR2 agonists, antagonists, or mixed agonist-antagonists). C) Measuring biochemical, molecular biological, cellular biological, physiological, and/or behavioral changes in said human subjects. D) Performing an analysis to determine if said changes represent statistically significant differences among various human subject groups; statistically significant differences from such analysis can validate the method for patient stratification into clinical trials.

The methods described herein have a number of applications. For example, diagnostic kits or assays are provided that include appropriate reagents to enable users to apply the methods of patient stratification into clinical trials, and/or into medical treatment options. Said appropriate reagents can include nucleic acids, proteins, lipids, polysaccharides, inorganic compounds, organic compounds, cellular extracts, intact cell types, synthetic molecules, and combinations thereof. The methods can be utilized as a turn-key service for the purpose of better predicting the treatment outcome of a CCR2 receptor compound. These methods can be used for new pharmaceutical drug development. In one example embodiment, one or more diagnostic kits and/or assays are provided that include determining cellular activities at the biochemical and/or physiological levels, in single cells, in cell-cell interactions, and/or in cell-free environment. In another example embodiment, one or more diagnostic kits and/or assays are provided that include a chemical indicator or indicators, such as fluorescent indicator compounds and/or luciferase markers, and the said chemical indicators' properties are measured. In yet another example embodiment, one or more diagnostic kits and/or assays are provided that include one or more chemical indicators, one or more cellular types, or a mixture of chemical indicators and cellular types, said substances are placed on a containment that consists of paper, cloth, plastic or other forms of polymers, metal, or a combination thereof, and said containment is enabled to be in contact with samples to be tested, so that said chemical indicators' properties and/or cellular activities can be determined. In yet another example embodiment, one or more diagnostic kits and/or assays are provided that include samples to be tested in a purified form, or in a mixture of liquid, solid, and/or gas state, and said samples to be tested are enabled to be in contact with a containment described above, for the purpose of activity measurement.

FIG. 6 is an illustration of a kit 100 for performing methods described herein. The kit 100 includes one or more reagents for use in performing methods described herein. While FIG. 6 illustrates a kit 100 with three reagents (Reagent A 110, Reagent B 111, and Reagent C 112), a kit 100 can include more or fewer reagents, as appropriate. In some embodiments, kit 100 can also include instructions 120 for performing methods described herein.

One type of kit is for performing polymerase chain reaction (PCR) for selectively amplifying CCR2A or CCR2B. The kit includes forward and reverse primers for performing PCR.

In one embodiment, the forward primer comprises (or is) SEQ ID NO: 7. In one embodiment, the forward primer comprises (or is) SEQ ID NO: 8.

In one embodiment, the reverse primer comprises (or is) SEQ ID NO: 18. In one embodiment, the reverse primer comprises (or is) SEQ ID NO: 17. In one embodiment, the reverse primer comprises (or is) SEQ ID NO: 22.

In one embodiment, the forward primer comprises (or is) SEQ ID NO: 7, and the reverse primer comprises (or is) SEQ ID NO: 18. In one embodiment, the forward primer comprises (or is) SEQ ID NO: 8, and the reverse primer comprises (or is) SEQ ID NO: 18. In one embodiment, the forward primer comprises (or is) SEQ ID NO: 8, and the reverse primer comprises (or is) SEQ ID NO: 22. In one embodiment, the forward primer comprises (or is) SEQ ID NO: 7, and the reverse primer comprises (or is) SEQ ID NO: 17. In one embodiment, the forward primer comprises (or is) SEQ ID NO: 8, and the reverse primer comprises (or is) SEQ ID NO: 17.

### Materials and Methods

### Human Blood Samples

Human blood samples were used in the study, with seventeen study subjects as listed in Table 1

TABLE 1
Medical status of patients

| Medical Status | Age | Sex |
|---|---|---|
| Diabetes | 49 | Male |
| Diabetes | 58 | Female |
| Diabetes | 62 | Male |
| Diabetic Neuropathy | 49 | Female |
| Diabetic Neuropathy | 52 | Female |
| Diabetic Neuropathy | 53 | Female |
| Diabetic Neuropathy | 60 | Male |
| Diabetic Neuropathy | 61 | Male |
| Diabetic Neuropathy | 61 | Male |
| Diabetic Neuropathy | 63 | Male |
| Diabetic Neuropathy | 77 | Female |
| Normal | 52 | Male |
| Normal | 54 | Male |
| Normal | 54 | Male |
| Normal | 55 | Female |
| Normal | 59 | Male |
| Normal | 68 | Female |

Total RNA was extracted from the 5 mL aliquots with the Monarch Total RNA Miniprep Kit (New England Biolabs) in conjunction with the Total RNA Purification from Mammalian Whole Blood Samples protocol provided by NEB (New England Biolabs). Extracted RNA samples were stored in aliquots at −80° C.

### PCR

Designed PCR primers were ordered from Integrated DNA Technologies, Inc. Primer formulation is considered to be Lab Use Ready stock (100 μM). For each study subject RNA, reverse transcription was performed according to the protocol provided by ThermoFischer Scientific with their TaqMan Reverse Transcription Reagents kit. Each reverse transcription reaction produced 20 μl of cDNA that was then used for subsequent PCR reaction. PCR was performed using the Taq DNA Polymerase with Standard Taq Buffer PCR kit supplied by New England Biolabs. The New England Biolabs PCR protocol that accompanied the kit was used to perform PCR. Upon completion of PCR, agarose gel electrophoresis was performed, and the gel stained with ethidium bromide in order to visualize the PCR products.

### Quantification of Splice Variant Expression Via Gel Densitometry

Gel densitometry was used to quantify isoform expression levels, performed with ImageJ, a Java-based image processing program. All CCR2 isoform PCR bands were compared against internal standard and/or GAPDH standard to allow for comparison across gel lanes. CCR2 isoform expression ratio was calculated based on the following equation: (CCR2B-CCR2A)/(CCR2B+CCR2A). The individual patient ratios were graphed and assessed qualitatively to determine whether the CCR2 isoform ratio changed across patients.

### Results

### Comparison of CCR2A Vs 2B Splice Variant Sequences

Prior to designing primers for CCR2A and CCR2B, we needed to understand the structure of the alternatively spliced variants. Research indicated that the protein sequences of the variants differed in their carboxyl terminals, which suggests that there is genetic sequence difference between the two splice variants. The nucleic acid sequence files for both human CCR2B and CCR2A were downloaded from NCBI website, and we performed sequence comparison. The sequence alignment depicted the regions where CCR2B and CCR2A overlapped.

Based on the sequence alignment, CCR2B contains an exon approximately around the middle section of the CCR2 nucleic acid sequence. Even though said exon results in a longer mRNA, differences in protein coding region results in CCR2A protein sequence longer than that for CCR2B.

### Designing Common and Splice Variant-Specific PCR Primers

Once we had a clear understanding of the splice variant sequences for CCR2A and CCR2B, we designed PCR primers both for common regions of CCR2A and CCR2B, as well as splice variant-specific PCR primers that would isolate the specific variants. Developing PCR primers specific and optimal to each of the variants was a necessary step that would allow efficient variant isolation needed to quantify the individual expression levels of CCR2A and CCR2B in blood samples from a human subject. This would allow us to analyze expression levels in subjects to determine whether the CCR2 splice variant ratio differs among different people.

We used the NCBI primer design tool to produce multiple primers specific for either CCR2A or CCR2B, as well as common to both. Our parameters for primer design included: 1) primer length within 20-40 nucleotides; 2) GC content of 40-60%; and 3) melting temperature (Tm) between 45-68° C. Primers lengths were all approximately 20 base pairs long and had similar melting temperatures. We designed primers close to exon boundaries, so that variant specificity could be measured. Primers designed for CCR2B were located within the CCR2B-specific exon. We designed primers for CCR2A that theoretically could also isolate CCR2B, but because the extra exon in CCR2B was over 1,000 bases longer than CCR2A, its amplification would be very low quantity, relatively compared to that of CCR2A.

We used GAPDH as our internal control, so as to quantify the PCR amplification product.

### Splice Variant Expression Levels

PCR products were analyzed by agarose gel electrophoresis, and the DNA bands were quantified by densitometry measurements using ImageJ software.

We used blood samples from normal healthy subjects, as well as diabetic patients with or without neuropathy. The results demonstrated that individual CCR2A/CCR2B ratios differ considerably among different study subjects, indicating that the CCR2 isoform ratios can serve as an indicator of a study subject's unique isoform levels, and that CCR2 isoform profiles can be used to stratify subject populations, for correlation with therapeutic treatment responders and non-responders.

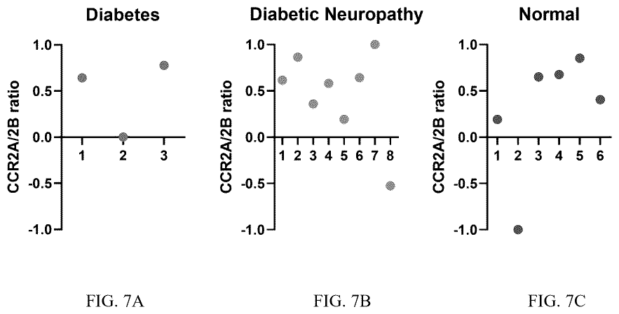

FIGS. 7A-C are graphs of ratios of human CCR2 isoform expression levels in human subjects, three with diabetes (FIG. 7A), eight with diabetic neuropathy (FIG. 7B), and six normal human subjects without diabetes-related disease (FIG. 7C).

### PCR Primer Pair Efficiency

PCR primer pair efficiency: For each primer pair, the primer pair code and forward/reverse primer sequences are shown. Column ‘CCR2A’ indicates the expected PCR product length in base pairs. Column ‘CCR2B’ indicates the expected PCR product length in base pairs. When a primer pair is capable of amplifying both a smaller CCR2A product and a larger CCR2B product, the expected CCR2B product would be 1,208 base pairs longer than that of CCR2A, due to the additional exon sequence contained in CCR2B isoform. This makes the amplification of the larger CCR2B product inefficient, to the extent that the expected larger product is not observed. Thus, the larger band size of expected product is shown in parenthesis. Column ‘Efficiency’ shows percentage number of PCR amplification efficiency compared to the internal control, for the shorter product.

| Primer Pair Code | Forward Primer | Reverse Primer | CCR2A | CCR2B | Efficiency |
|---|---|---|---|---|---|
| CD | SEQ ID NO: 7 | SEQ ID NO: 16 | 453 | (1,661) | 22% |
| CH | SEQ ID NO: 7 | SEQ ID NO: 17 | — | 315 | 57% |
| CJ | SEQ ID NO: 7 | SEQ ID NO: 18 | 422 | (1,630) | 50% |
| CL | SEQ ID NO: 7 | SEQ ID NO: 19 | 513 | (1,721) | 13% |
| CM | SEQ ID NO: 7 | SEQ ID NO: 20 | 311 | (1,519) | 0% |
| CN | SEQ ID NO: 7 | SEQ ID NO: 21 | 360 | (1,568) | 15% |
| CO | SEQ ID NO: 7 | SEQ ID NO: 22 | 372 | (1,580) | 0% |
| ID | SEQ ID NO: 8 | SEQ ID NO: 16 | 360 | (1,568) | 25% |
| IH | SEQ ID NO: 8 | SEQ ID NO: 17 | — | 222 | 69% |
| IJ | SEQ ID NO: 8 | SEQ ID NO: 18 | 329 | (1,537) | 50% |
| IL | SEQ ID NO: 8 | SEQ ID NO: 19 | 420 | (1,628) | 22% |
| IM | SEQ ID NO: 8 | SEQ ID NO: 20 | 218 | (1,426) | 31% |
| IN | SEQ ID NO: 8 | SEQ ID NO: 21 | 267 | (1,475) | 27% |
| IO | SEQ ID NO: 8 | SEQ ID NO: 22 | 279 | (1,487) | 50% |
| KL | SEQ ID NO: 9 | SEQ ID NO: 19 | 548 | (1,756) | 0% |
| PQ | SEQ ID NO: 10 | SEQ ID NO: 23 | 218 | 218 | 46% |
| RV | SEQ ID NO: 11 | SEQ ID NO: 25 | 232 | 232 | 37% |

For selectively amplifying CCR2A, preferred primer pairs are CJ, IJ, and IO. For selectively amplifying CCR2B, preferred primer pairs are CH and IH.

### INDEX OF SEQUENCES

Table 2 provides an indexes of the sequences included with the sequence listing.

TABLE 2
Index of sequence listing

| SEQ ID NO | Description |
|---|---|
| SEQ ID NO: 1 | CCR2 isoform A cDNA |
| SEQ ID NO: 2 | CCR2 isoform A mRNA |
| SEQ ID NO: 3 | CCR2 isoform A protein |
| SEQ ID NO: 4 | CCR2 isoform B cDNA |
| SEQ ID NO: 5 | CCR2 isoform B mRNA |
| SEQ ID NO: 6 | CCR2 isoform B protein |
| SEQ ID NO: 7 | Forward primer; Primer code: C |
| SEQ ID NO: 8 | Forward primer; Primer code: I |
| SEQ ID NO: 9 | Forward primer; Primer code: K |
| SEQ ID NO: 10 | Forward primer; Primer code: P |
| SEQ ID NO: 11 | Forward primer; Primer code: R |
| SEQ ID NO: 12 | Forward primer; Primer code: T |
| SEQ ID NO: 13 | Forward primer; Primer code: W |
| SEQ ID NO: 14 | Forward primer; Primer code: Y |
| SEQ ID NO: 15 | Forward primer; Primer code: APF |
| SEQ ID NO: 16 | Reverse primer: Primer code: D |
| SEQ ID NO: 17 | Reverse primer: Primer code: H |
| SEQ ID NO: 18 | Reverse primer: Primer code: J |
| SEQ ID NO: 19 | Reverse primer: Primer code: L |
| SEQ ID NO: 20 | Reverse primer: Primer code: M |
| SEQ ID NO: 21 | Reverse primer: Primer code: N |
| SEQ ID NO: 22 | Reverse primer: Primer code: O |
| SEQ ID NO: 23 | Reverse primer: Primer code: Q |
| SEQ ID NO: 24 | Reverse primer: Primer code: U |
| SEQ ID NO: 25 | Reverse primer: Primer code: V |
| SEQ ID NO: 26 | Reverse primer: Primer code: X |
| SEQ ID NO: 27 | Reverse primer: Primer code: Z |
| SEQ ID NO: 28 | Reverse primer: Primer code: APR |

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes, modifications, and enhancements in form and details may be made therein without departing from the scope of the embodiments encompassed by the appended claims.

What is claimed is:

1. A method for treating a disease in a patient in need thereof, the method comprising:
a) measuring a CCR2 profile of the patient, the CCR2 profile comprising a profile of CCR2 isoform A, CCR2 isoform B, or both, each CCR2 isoform comprising one or more respective polymorphisms;
b) selecting a compound based on the CCR2 profile of the patient, wherein the selected compound is an antagonist or agonist of at least one polymorphism identified from the measured CCR2 profile of the patient;
c) administering the selected compound to the patient.

2. The method of claim 1, wherein measuring the CCR2 profile comprises sequencing CCR2 proteins or nucleic acids within the patient.

3. The method of claim 2, wherein measuring the CCR2 profile comprises sequencing nucleic acids within the patient.

4. The method of claim 3, wherein the nucleic acids are DNA.

5. The method of claim 3, wherein the nucleic acids are mRNA.

6. The method of claim 5, wherein the mRNA is within a bodily fluid of the patient.

7. The method of claim 6, wherein the mRNA is within blood.

8. The method of any one of claims 2-7, further comprising separating a biological material based on the sequenced proteins or nucleic acids, wherein the biological material comprises cells, tissue, blood, other bodily fluids, or a combination thereof.

9. The method of any one of claims 1-8, wherein measuring the CCR2 profile of the patient comprises measuring a CCR2 profile of one or any combination of the patient's cells, the patient's tissues, the patient's blood, and other bodily fluids of the patient.

10. The method of claim 9, wherein measuring the CCR2 profile of the patient comprises measuring a CCR2 profile of the patient's cells, optionally wherein the cells are a plurality of cell types.

11. The method of claim 9, wherein measuring the CCR2 profile of the patient of the patient comprises measuring a CCR2 profile of the patient's tissues, optionally wherein the tissues are a plurality of tissue types.

12. The method of claim 9, wherein measuring the CCR2 profile of the patient comprises measuring a CCR2 profile of the patient's other bodily fluids, optionally wherein the other bodily fluids are a plurality of types of bodily fluids.

13. The method of any one of claims 1-12, wherein measuring the CCR2 profile of the patient comprises measuring a protein expression level for CCR2 isoform A and CCR2 isoform B, or both.

14. The method of claim 13, further comprising separating a biological material based on the measured protein expression level for CCR2 isoform A, CCR2 isoform B, or both, wherein the biological material comprises cells, tissue, blood, other bodily fluids, or a combination thereof.

15. The method of any one of claims 1-14, wherein measuring a CCR2 profile comprises subjecting a biological material to one or more compounds, wherein the biological material comprises cells, tissue, blood, other bodily fluids, or a combination thereof, wherein the one or more compounds are antagonists or agonists of at least one polymorphism identified from the measured CCR2 profile of the patient.

16. The method of claim 15, further comprising measuring one or more of biochemical, molecular biological, and cellular biological changes in the biological material prior to and after subjecting the biological material to the one or more compounds.

17. The method of any one of claims 1-16, wherein measuring a CCR2 profile comprises obtaining a biological sample from the patient.

18. The method of any one of claims 1-17, wherein the selected compound is one or more of INCB-8761/PF-4136309, MK-0812, BMS-813160, INCB-003284, PF-04634817, BMS-741672, Cenicriviroc (CCR2+CCR5), and CCX-140.

19. The method of any one of claims 1-18, wherein the selected compound is an antagonist of at least one polymorphism of CCR2 isoform A identified from the measured CCR2 profile of the patient.

20. The method of any one of claims 1-18, wherein the selected compound is an agonist of at least one polymorphism of CCR2 isoform A identified from the measured CCR2 profile of the patient.

21. The method of any one of claims 1-18, wherein the selected compound is an antagonist of at least one polymorphism of CCR2 isoform B identified from the measured CCR2 profile of the patient.

22. The method of any one of claims 1-18, wherein the selected compound is an agonist of at least one polymorphism of CCR2 isoform B identified from the measured CCR2 profile of the patient.

23. The method of any one of claims 1-18, wherein the selected compound is one of either an antagonist or an agonist for each of a plurality of polymorphisms of CCR2 isoform A identified from the measured CCR2 profile of the patient.

24. The method of any one of claims 1-18, wherein the selected compound is an antagonist for each of a plurality of polymorphisms of CCR2 isoform A identified from the measured CCR2 profile of the patient.

25. The method of any one of claims 1-18, wherein the selected compound is an agonist for each of a plurality of polymorphisms of CCR2 isoform A identified from the measured CCR2 profile of the patient.

26. The method of any one of claims 1-18, wherein the selected compound is one of either an antagonist or agonist for each of a plurality of polymorphisms of CCR2 isoform B identified from the measured CCR2 profile of the patient.

27. The method of any one of claims 1-18, wherein the selected compound is an antagonist for each of a plurality of polymorphisms of CCR2 isoform B identified from the measured CCR2 profile of the patient.

28. The method of any one of claims 1-18, wherein the selected compound is an agonist for each of a plurality of polymorphisms of CCR2 isoform B identified from the measured CCR2 profile of the patient.

29. The method of any one of claim 1-18, wherein the selected compound is an agonist of at least one polymorphism of CCR2 isoform A identified from the measured CCR2 profile of the patient and an antagonist of at least one polymorphism of CCR2 isoform B identified from the measured CCR2 profile of the patient.

30. The method of any one of claim 1-18, wherein the selected compound is an antagonist of at least one polymorphism of CCR2 isoform A identified from the measured CCR2 profile of the patient and an agonist of at least one polymorphism of CCR2 isoform B identified from the measured CCR2 profile of the patient.

31. A method for stratifying one or more patients for pain medication based on a CCR2 profile, the method comprising:
a) measuring a CCR2 profile of one or more patients, the CCR2 profile comprising a profile of CCR2 isoform A, CCR2 isoform B, or both, each CCR2 isoform comprising one or more respective polymorphism; and
b) partitioning the one or more patients into predetermined groups or subgroups based on the CCR2 profile of the one or more patients.

32. The method of claim 31, wherein measuring the CCR2 profile comprises sequencing CCR2 proteins or nucleic acids within the patient.

33. The method of claim 32, wherein measuring the CCR2 profile of the one or more patients comprises sequencing nucleic acids within the patient.

34. The method of claim 33, wherein the nucleic acids are DNA.

35. The method of claim 33, wherein the nucleic acids are mRNA.

36. The method of claim 35, wherein the mRNA is within a bodily fluid of the patient.

37. The method of claim 36, wherein the mRNA is within blood.

38. The method of any one of claims 32-37, further comprising separating a biological material based on the sequenced proteins or nucleic acids, wherein the biological material comprises cells, tissue, blood, other bodily fluids, or a combination thereof.

39. The method of any one of claims 31-38, wherein measuring the CCR2 profile of the one or more patients comprises measuring a CCR2 profile of one or any combination of the patient's cells, the patient's tissues, the patient's blood, and other bodily fluids of the patient.

40. The method of claim 39, wherein measuring the CCR2 profile of the one or more patients comprises measuring a CCR2 profile of the patient's cells, optionally wherein the cells are a plurality of cell types.

41. The method of claim 39, wherein measuring the CCR2 profile of the one or more patients comprises measuring a CCR2 profile of the patient's tissues, optionally wherein the tissues are a plurality of tissue types.

42. The method of claim 39, wherein measuring the CCR2 profile of the one or more patients comprises measuring a CCR2 profile of the patient's other bodily fluids, optionally wherein the other bodily fluids are a plurality of types of bodily fluids.

43. The method of any one of claims 31-42, wherein measuring the CCR2 profile of the one or more patients comprises measuring a protein expression level for CCR2 isoform A, CCR2 isoform B, or both.

44. The method of claim 43, further comprising separating a biological material based on the measured protein expression level for CCR2 isoform A, CCR2 isoform B, or both, wherein the biological material comprises cells, tissue, blood, other bodily fluids, or a combination thereof.

45. The method of any one of claims 31-44, wherein measuring a CCR2 profile comprises subjecting a biological material to one or more compounds, wherein the biological material comprises cells, tissue, blood, other bodily fluids, or a combination thereof, wherein the one or more compounds are antagonists or agonists of at least one polymorphism of identified from the measured CCR2 profile of the patient.

46. The method of claim 45, further comprising measuring one or more of biochemical, molecular biological, and cellular biological changes in the biological material prior to and after subjecting the biological material to the one or more compounds.

47. The method of claim 46, wherein the one or more compounds is one or more of INCB-8761/PF-4136309, MK-0812, BMS-813160, INCB-003284, PF-04634817, BMS-741672, Cenicriviroc (CCR2+CCR5), and CCX-140.

48. The method of any one of claims 31-47, wherein measuring a CCR2 profile comprises obtaining a biological sample from the one or more patients.

49. The method of any one of claims 31-48, further comprising identifying statistically significant differences among patient groups or subgroups.

50. The method of any one of claims 45-46, wherein the compound is an antagonist of at least one polymorphism of CCR2 isoform A identified from the measured CCR2 profile of the patient.

51. The method of any one of claims 45-46, wherein the compound is an agonist of at least one polymorphism of CCR2 isoform A identified from the measured CCR2 profile of the patient.

52. The method of any one of claims 45-46, wherein the compound is an antagonist of at least one polymorphism of CCR2 isoform B identified from the measured CCR2 profile of the patient.

53. The method of any one of claims 45-46, wherein the compound is an agonist of at least one polymorphism of CCR2 isoform B identified from the measured CCR2 profile of the patient.

54. The method of any one of claims 45-46, wherein the compound is one of either an antagonist or an agonist for each of a plurality of polymorphisms of CCR2 isoform A identified from the measured CCR2 profile of the patient.

55. The method of any one of claims 45-46, wherein the compound is an antagonist for each of a plurality of polymorphisms of CCR2 isoform A identified from the measured CCR2 profile of the patient.

56. The method of any one of claims 45-46, wherein the compound is an agonist for each of a plurality of polymorphisms of CCR2 isoform A identified from the measured CCR2 profile of the patient.

57. The method of any one of claims 45-46, wherein the compound is one of either an antagonist or agonist for each of a plurality of polymorphisms of CCR2 isoform B identified from the measured CCR2 profile of the patient.

58. The method of any one of claims 45-46, wherein the compound is an antagonist for each of a plurality of polymorphisms of CCR2 isoform B identified from the measured CCR2 profile of the patient.

59. The method of any one of claims 45-46, wherein the compound is an agonist for each of a plurality of polymorphisms of CCR2 isoform B identified from the measured CCR2 profile of the patient.

60. The method of any one of claim 45-46, wherein the compound is an agonist of at least one polymorphism of CCR2 isoform A identified from the measured CCR2 profile of the patient and an antagonist of at least one polymorphism of CCR2 isoform B identified from the measured CCR2 profile of the patient.

61. The method of any one of claim 45-46, wherein the compound is an antagonist of at least one polymorphism of CCR2 isoform A identified from the measured CCR2 profile of the patient and an agonist of at least one polymorphism of CCR2 isoform B identified from the measured CCR2 profile of the patient.

62. A kit for performing any one of the methods described in claims 1-30, the kit comprising:
a) one or more reagents for measuring a CCR2 profile of the patient, the CCR2 profile comprising a profile of CCR2 isoform A, CCR2 isoform B, or both, each CCR2 isoform comprising one or more respective polymorphisms.

63. A kit for performing any one of the methods described in claims 31-61, the kit comprising:
a) one or more reagents for measuring a CCR2 profile of the patient, the CCR2 profile comprising a profile of CCR2 isoform A, CCR2 isoform B, or both, each CCR2 isoform comprising one or more respective polymorphisms, wherein the one or more reagents comprises a forward primer comprising a sequence selected from SEQ ID NOs: 7 through 15 and a reverse primer comprising a sequence selected from SEQ ID NOs: 16 through 28.

64. The kit of claim 63, wherein reagents for measuring a CCR2 isoform A profile comprise a forward primer comprising SEQ ID NO: 7 and a reverse primer comprising SEQ ID NO: 18.

65. The kit of claim 63, wherein reagents for measuring a CCR2 isoform A profile comprise a forward primer comprising SEQ ID NO: 8 and a reverse primer comprising SEQ ID NO: 18.

66. The kit of claim 63, wherein reagents for measuring a CCR2 isoform A profile comprise a forward primer comprising SEQ ID NO: 8 and a reverse primer comprising SEQ ID NO: 22.

67. The kit of claim 63, wherein reagents for measuring a CCR2 isoform B profile comprise a forward primer comprising SEQ ID NO: 7 and a reverse primer comprising SEQ ID NO: 17.

68. The kit of claim 63, wherein reagents for measuring a CCR2 isoform B profile comprise a forward primer comprising SEQ ID NO: 8 and a reverse primer comprising SEQ ID NO: 17.

69. A method for generating a profile of CCR2A and CCR2B isoform expression levels for a human subject, the method comprising:
a) obtaining a sample from the human subject;
b) extracting RNA from the sample;
c) performing reverse transcription on the extracted RNA to generate cDNA;
d) amplifying the cDNA by performing polymerase chain reaction (PCR) by using a forward primer comprising a sequence selected from SEQ ID NOs: 7 through 15 and a reverse primer comprising a sequence selected from SEQ ID NOs: 16 through 28; and
e) quantifying CCR2A and CCR2B isoform expression levels from the amplified CDNA.

70. The method of claim 69, wherein the forward primer comprises SEQ ID NO: 7.

71. The method of claim 69, wherein the forward primer comprises SEQ ID NO: 8.

72. The method of claim 69, wherein the reverse primer comprises SEQ ID NO: 18.

73. The method of claim 69, wherein the reverse primer comprises SEQ ID NO: 17.

74. The method of claim 69, wherein the reverse primer comprises SEQ ID NO: 22.

75. The method of claim 69, wherein the forward primer comprises SEQ ID NO: 7, and wherein the reverse primer comprises SEQ ID NO: 18.

76. The method of claim 69, wherein the forward primer comprises SEQ ID NO: 8, and wherein the reverse primer comprises SEQ ID NO: 18.

77. The method of claim 69, wherein the forward primer comprises SEQ ID NO: 8, and wherein the reverse primer comprises SEQ ID NO: 22.

78. The method of claim 69, wherein the forward primer comprises SEQ ID NO: 7, and wherein the reverse primer comprises SEQ ID NO: 17.

79. The method of claim 69, wherein the forward primer comprises SEQ ID NO: 8, and wherein the reverse primer comprises SEQ ID NO: 17.

80. The method of claim 69, wherein quantifying CCR2A and CCR2B isoform expression levels is by gel densitometry, blotting, nucleic acid hybridization, fluorescent in situ hybridization, reverse transcription-polymerase chain reaction, real-time polymerase chain reaction, microarray, nucleic acid sequencing, enzyme-linked immunosorbent assay (ELISA), or colorimetric quantification.

81. The method of claim 69, wherein the sample is blood.

82. The method of claim 69, wherein the sample is a bodily secretion.

83. The method of claim 82, wherein the bodily secretion is saliva.

84. The method of claim 82, wherein the bodily secretion is urine.

85. The method of claim 82, wherein the bodily secretion is nasal mucosa.

86. The method of claim 82, wherein the bodily secretion is vaginal mucosa.
